(19) United States
(12) Patent Application Publication
(10) Pub. No.: US 2007/0134173 A1
(43) Pub. Date: Jun. 14, 2007

(54) Personal care compositions

(75) Inventors: Shuhei Tanaka, Higashinaka-Ku (JP); Kiyoaki Mori, Yasu-Shi (JP)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company, Cincinnati, OH (US)

(21) Appl. No.: 11/298,005

(22) Filed: Dec. 9, 2005

(51) Int. Cl. A61K 8/37; A61K 31/375; A61K 31/51

(52) U.S. Cl. 424/59; 424/63; 514/474; 514/276

(57) ABSTRACT

Methods for stabilizing color of a personal care composition containing a skin care active that is unstable in oxidizing medium are provided. In one of the preferred embodiments, the method includes the step of incorporating into the composition a color stabilizing system including a carotenoid and an ultraviolet light absorbing agent. Color stable personal care compositions are also provided.

### FIELD OF THE INVENTION

The present invention relates to methods for stabilizing the color of personal care compositions including an active that is unstable in oxidizing medium. The present invention further relates to color stable personal care compositions including such actives.

### BACKGROUND OF THE INVENTION

It is known to introduce active principles into personal care compositions for the purpose of contributing specific treatments to the skin and/or hair. The treatments include, for example, cleansing skin, combating drying, ageing, or pigmentation of the skin, treating acne or other skin disorder such as eczema and psoriasis, and promoting restructuring of the skin or its cell replacement.

Vitamin C (e.g., ascorbyl glucoside) is one known skin care active. It is believed to stimulate collagen synthesis, strengthen cutaneous tissues against external attack (UV radiation, pollution), and lighten and/or whiten the skin. Vitamin C is effective for cleansing the skin and also in combating signs of ageing of the skin, for example, in improving complexion, and softening fine lines and wrinkles. Vitamin A (e.g., retinol) is another known skin care active for regulating or treating keratinous tissue.

Unfortunately, these active principles can be unstable to various degrees in oxidizing medium. The actives are therefore sensitive to certain environmental parameters, such as, for example, light, air, oxygen, heat and water. Oxidation of the active reduces the amount of the active available within a composition that can lead to a corresponding reduction in desired effectiveness. Furthermore, oxidation of an active can undesirably discolor the composition, for example, from whitish to yellowish. Consumers may disfavor applying a yellow-hued product on their skin, particularly if the composition is for cleansing or intended to make the skin look younger and healthier. Moreover, consumers may perceive a color-changed product to have “gone bad.”

### SUMMARY OF PREFERRED EMBODIMENTS

The present invention provides methods for stabilizing the color of personal care compositions including an active that is unstable in oxidizing medium. In accordance with one of the preferred embodiments, there has now been provided a method comprising the step of incorporating into the composition a color stabilizing system comprising a carotenoid and an ultraviolet light absorbing agent. In another preferred embodiment, the color stabilizing system further comprises an inorganic pigment. In yet another preferred embodiment the color stabilizing system further comprises an antioxidant.

In accordance with another preferred method embodiment, there has now been provided a method for stabilizing color of a personal care composition that turns yellow when exposed to oxidizing medium by incorporating into the composition 1) a natural yellow, orange, red, and/or brown pigment; and 2) two or more materials that inhibit oxidation selected from the group comprising anti-oxidants, inorganic pigments, ultraviolet A absorbing agents, and ultraviolet B absorbing agents.

The present invention also provides color stable personal care compositions that include an active that is unstable in oxidizing medium. Preferred compositions include, for example, water-in-oil and oil-in-water emulsions, and lotion and cleansing cosmetic products. In accordance with one of the preferred embodiments, there has now been provided a personal care composition including an active that is unstable in oxidizing medium, a multi-component color stabilizing system, and a dermatologically acceptable carrier. The multi-component color stabilizing system comprises a carotenoid, an anti-oxidant, and a UVB absorbing agent.

In accordance with another preferred embodiment, there has now been provided a personal care composition including an active that is unstable in oxidizing medium, and a multi-component color stabilizing system comprising a carotenoid and an ultraviolet light absorbing agent.

These and various other features of novelty, and their respective advantages, are pointed with particularity in the claims annexed hereto and forming a part hereof.

### DETAILED DESCRIPTION OF ILLUSTRATIVE EMBODIMENTS

The present invention may be understood more readily by reference to the following detailed description of illustrative and preferred embodiments. It is to be understood that the scope of the claims is not limited to the specific methods, conditions or parameters described herein, and that the terminology used herein is for the purpose of describing particular embodiments by of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. When a range of values is expressed, another embodiment includes from the one particular value and/or to the other particular value. Similarly, when values are expressed as approximations, by use of the antecedent “about” or “substantially,” it will be understood that the particular value forms another embodiments. All ranges are inclusive and combinable.

I. Illustrative Methods

The present invention provides methods for stabilizing the color of personal care compositions that contain an active which is unstable in oxidizing medium, such as light, air, oxygen, heat and water. The preferred methods include incorporating a color stabilizing system into the personal care composition. The color stabilizing systems contemplated herein generally comprise one or more materials that inhibit oxidation of the active and/or mask the discoloration associated with oxidation of the active. Other mechanisms may be utilized alternatively or additionally to anti-oxidation and masking. Preferred systems comprise multiple materials to provide color stabilizing benefits (“multi-component color stabilizing system” via one or more mechanisms). By way of example, color stabilizing systems can include ultraviolet light absorbing agents, colorants, and anti-oxidants.

A. Ultraviolet Light Absorbing Agents The color stabilizing systems may include one or more ultraviolet-A (“UVA”) and/or ultraviolet-B (“UVB”) absorbing agents. When present, the ultraviolet light absorbing agents are preferably included at a level of from about 0.01% by weight to about 20% by weight, more preferably from about 0.5% by weight to about 10% by weight, and even more preferably from about 1% by weight to about 4% by weight, based on the total weight of the personal care compositions. Other inclusion levels are within the scope of appended claims that explicitly include ultraviolet light absorbing agents but do not otherwise recite a specific range.

A representative, non-limiting list of suitable ultraviolet light absorbing agents includes octyl methoxycinnamate, oxybenzone, octocrylene, 2-ethylhexyl p-methoxycinnamate, 1-p-aminobenzoate, p-aminovbenzoic acid, 2-phenylbenzimidazole-5-sulfonic acid, homomenthyl salicylate, octyl salicylate, 4,4′-methoxy-t-butyldibenzoylmethane, 4-isopropyl dibenzoylmethane, 3-(4-methylbenzyldene)camphor, 3-benzylidenecamphor, 2-ethylhexyl 4-(dimethylamino)benzoate, amyl 4-(dimethylamino)benzoate, 2-ethylhexyl 4-methoxycinnamate, isopentyl 4-methoxycinnamate, 2-ehtylhexyl salicylate, 4-isopropylbenzyl salicylate, homomenthyl salicylate, 2-hydroxy-4-methoxybenzophenone, 2-hydroxy-4-methoxy-4′-methylbenzophenone, 2,2′-dihydroxy-4-methoxybenzophenone, di(2-ethylhexyl) 4-methoxybenzalmalonate, 2,4,6-trianilio(p-carbo-2′-ethyl-1′-hexyloxy)1,3,5-triazine, salts of 2-phenylbenzimadole-5-sulphonic acid, sulphonic acid derivatives of benzophenones, sulphonic acid derivatives of 3-benzylidenecamphor, 1(4′-tert-butylphenyl)-3-(4′methoxyphenyl)propane-1,3-dione, and 1-phnyl-3-(4′-isopropylphenyl)propane-1,3-dione.

Octyl methoxycinnamate (also known as octinoxate) is one preferred ultraviolet light absorbing agent. Inclusion levels of octyl methoxycinnamte preferably include from about 0.5% by weight to about 4% by weight, and more preferably from about 1% by weight to about 2% by weight, based on the total weight of the personal care compositions. Suitable octyl methoxycinnamate materials are commercially available from Matsumoto Kosho of Japan.

In one preferred embodiment, only a UVB absorbing agent is employed, while a UVA absorbing agent is not used.

B. Colorants

The color stabilizing systems may include one or more colorants. “Colorants” as used herein includes, but is not limited to, opacifiers, organic pigments, inorganic pigments, interference pigments, lakes, natural colorants/pigments, artificial colorants/pigments, pearlescent agents, dyes, carmines, and mixtures thereof. Preferred inclusion ranges of these materials, when present, are from about 0.0001% by weight to about 30% by weight, from about 0.001% by weight to about 5% by weight, and from about 0.01% by weight to about 1% by weight, based on the total weight of the personal care compositions. Other inclusion levels are within the scope of appended claims that include colorants but do not otherwise recite a specific range. Opacifiers may be included to block light-inducing oxidation of the personal care compositions. Opacifiers can also serve as pigments. For example, titanium dioxide and zinc oxide can both block light, and also provide the composition into which they are incorporated a white appearance. White opacifiers/pigments can be particularly desired in personal care compositions targeted for regulating skin tone or color, as application of such compositions can provide an acute skin whitening/lightening benefit to the user.

Titanium dioxide is one preferred opacifier in accordance with the present invention. Embodiments including titanium dioxide preferably have from about 0.1% by weight of the composition to about 1% by weight of the composition. Applicants have discovered that titanium dioxide materials having smaller and more homogenous size particles may be more effective. By way of example only, a titanium dioxide material having an average particle size of 0.3 microns may offer better color stability as compared to a titanium dioxide material having an average particle size in the range of 1 to 5 microns. Notwithstanding this discovery, titanium dioxide and other opacifiers having an average particle size that is smaller than 0.3 microns and larger than 5 microns are equally suitable for the present invention. One suitable titanium dioxide is product code CM3FA70STC commercially available from Kobo Products, Inc. of South Plainfield, N.J. Opacifiers, such as, titanium dioxide, are preferably coated with a non-metallic material, such as, for example, a silicone material, to minimize any reaction with the unstable active. Another suitable coating material is aluminum stearate.

Colorants can be employed to mask discoloration of personal care compositions that include an active that is unstable in oxidizing medium. For example, oxidation of vitamin C can cause a personal care composition containing the same to turn yellowish. Titanium dioxide or other white-colored colorants can be used to maintain a composition's original white appearance by masking such yellowing.

Another masking approach contemplated herein is to include natural or artificial colorants (including materials that are not necessarily known or classified as pigments, dyes, etc.,) that have the same color as that manifested through oxidation of the unstable active. One preferred class of natural colorants is carotenoids, which includes, for example, beta-carotene and lycopene. Carotenoids are generally considered any of a group of yellow, orange, red, or brown pigments found in many living organisms, including plants and animals. Continuing with the example of vitamin C above, a colorant material that portrays a yellow hue (e.g., beta-carotene) can be incorporated into the personal care compositions. The composition containing vitamin C will accordingly manifest a yellowish appearance when manufactured and exhibit more consistent color overtime even with the existence of vitamin C oxidation.

Natural and artificial colorants themselves can discolor when exposed to oxidizing medium, such as light. The discoloration is typically in the form of fading (e.g., a reduction in hue intensity or brightness). Applicants have discovered that the color appearance of a personal care composition can be substantially maintained overtime by incorporating an amount of a colorant that effectively balances/matches the increase in discoloration due to oxidation of an unstable active with the fading or decrease in intensity/brightness of the colorant. An exemplary method includes incorporating beta-carotene into a personal care composition containing vitamin C. Oxidation of the vitamin C increases the yellow appearance of the composition, and the intensity of the beta-carotene fades during a similar timeframe. This results in the composition color staying substantially the same through a lengthened shelf and use life. A Macbeth color measurement device, or the like, can be used to measure one or multi-dimensional color changes—delta b is the level of yellowness measured by a Macbeth device. Note that the rate of discoloration due to oxidation and the rate of colorant fading can be substantially the same or can be different. Where the rates differ, delta b measurements at different intervals may yield positive or negative values (or increase or decrease from one measurement interval to the next), with an overall objective being to control the average delta b value preferably around zero over the product's targeted shelf and use life.

Non-limiting examples of colorants include: carotenoids, D&C Red 30 Talc Lake, D&C Red 7 Calcium Lake, D&C Red 34 Calcium Lake, mica/titanium dioxide/carmine pigments (e.g., Clorisonne Red from Engelhard, Duocrome RB from Engelhard, Magenta from Rona, Dichrona RB from Rona), Red 30 low iron, D&C Red Lake blend of Lake 27 & Lake 30, FD&C Yellow 5 Lake, Kowet titanium dioxide, yellow iron oxide, D&C Red 30 Lake, D&C Red 28 Lake, Cos Red Oxide BC, Cos Iron Oxide Red BC, Cos Iron Oxide Yellow, Cos Iron Oxide Yellow BC, Euroxide Red Unsteril, Euroxide Yellow Steril, Euroxide Red, Hydrophobic Euroxide Yellow, Hydrophobic Euroxide Red, D&C Yellow 6 lake, D&C Yellow 5 Zr Lake, and mixtures thereof.

C. Anti-oxidants

The color stabilizing systems of the present invention may include one or more anti-oxidants. When present, the anti-oxidants are preferably included in an amount from about 0.001% by weight to about 5% by weight, more preferably included in an amount from about 0.01% by weight to about 3% by weight, and even more preferably included in an amount of from about 0.015% by weight to about 0.05% by weight, based on the total weight of the personal care composition. Other inclusion levels may also be used.

Examples of anti-oxidants include, but are not limited to, water-soluble anti-oxidants such as sulfhydryl compounds and their derivatives (e.g., sodium metabisulfite and N-acetyl-cysteine), lipoic acid, and dihydrolipoic acid, resveratrol, lactoferrin, ascorbic acid, and ascorbic acid derivatives (e.g., ascorbyl palmitate and ascorbyl polypeptide). Oil-soluble anti-oxidants include, but are not limited to, butylated hydroxytoluene, retinoids (e.g., retinol and retinyl palmitate), tocopherols (e.g., tocopheryl acetate), tocotrienols, and ubiquionone. Sodium metabisulfite is one preferred anti-oxidant, preferably included at a level of from about 0.1% by weight of the total composition to about 5% by weight of the total composition.

As noted above, various combinations of these classes of color stabilizing materials can be incorporated into personal care compositions. Additional classes/types of materials may also be included in the color stabilizing systems in accordance with the present invention. In one preferred method embodiment, a color stabilizing system comprising the combination of octyl methoxycinnamate, titanium dioxide, and beta-carotene is incorporated into a personal care composition containing an active that is unstable in oxidizing medium. In another preferred method embodiment, a color stabilizing system comprising the combination of octyl methoxycinnamate, titanium dioxide, beta-carotene, and sodium metabisulfite is incorporated into a personal care composition containing an active that is unstable in oxidizing medium.

II. Illustrative Compositions

Compositions of the present invention contain a safe and effective amount of one or more actives and a color stabilizing system as described supra.

As used herein, “safe and effective amount” means an amount that is sufficient to induce a positive modification in the condition to be regulated or treated, while avoiding serious side effects; i.e., to provide a reasonable benefit to risk ratio, within the scope of sound judgment of the skilled artisan. The safe and effective amount of an active may vary with the age and physical condition of the user, the skin and hair type of the user, the severity of the condition being regulated or treated, the duration of the treatment, the nature of any concurrent therapy, and like factors.

The compositions of the present invention can include, consist essentially of, or consist of, the components of the present invention as well as other ingredients described herein. As used herein, “consisting essentially of” means that the composition or component may include additional ingredients, but only if the additional ingredients do not materially alter the basic and novel characteristics of the claimed compositions or methods.

All percentages, parts and ratios are based upon the total weight of the personal care compositions of the present invention, unless otherwise specified. All such weights as they pertain to listed ingredients are based on the active level and, therefore, do not include carriers or by-products that may be included in commercially available materials, unless otherwise specified.

Preferred composition embodiments include at least one active that is unstable in oxidizing medium—for example, vitamin C, vitamin A, or derivatives thereof. Other unstable actives may also be employed.

One exemplary composition includes vitamin C and/or its derivatives, which comprises, inter alia, ascorbic acid esters such as sodium ascorbate, magnesium ascorbate phosphate, ascorbyl aminopropyl phosphate, 2-sodium ascorbate sulfate ester, sodium ascorbate phosphate ester, ascorbate state ester, ascorbate palmitate ester, ascorbate diplamitate ester, and ascorbic acid ethers such as ascorbyl glucoside, ascorbate-2-O-α-glucoside, ethoxylated ascorbic acid, ascorbyl tetra-2-hexyldecanate, and ascorbyl glucosamine.

Another exemplary composition includes vitamin A and/or its derivatives, such as, a retinoid. As used herein, “retinoid” includes all natural and/or synthetic analogs of Vitamin A or retinol-like compounds which possess the biological activity of Vitamin A in the skin as well as the geometric isomers and stereoisomers of these compounds. The retinoid is preferably selected from retinol, retinol esters (e.g., C2-C22 alkyl esters of retinol, including retinyl palmitate, retinyl acetate, retinyl propionate), retinal, and/or retinoic acid (including all-trans retinoic acid and/or 13-cis-retinoic acid), or mixtures thereof.

The compositions of the present invention typically contain a dermatologically acceptable carrier within which other components in the composition are incorporated in order to enable the other components to be delivered at an appropriate concentration. The term “dermatologically-acceptable,” as used herein, means that the compositions or components thereof so described are suitable for use in contact with mammalian keratinous tissue without undue toxicity, incompatibility, instability, allergic response, and the like.

The carrier may contain one or more dermatologically acceptable solid, semi-solid or liquid fillers, diluents, solvents, extenders and the like. The carrier may be solid, semi-solid or liquid. The carrier can itself be inert or it can possess dermatological benefits of its own. Concentrations of the carrier can vary with the carrier selected and the intended concentrations of the composition components.

The type of carrier utilized in the present invention depends on the type of product form desired for the composition. The compositions of the present invention may be made into a wide variety of product forms such as are known in the art. These include, but are not limited to, lotions, creams, gels, sticks, sprays, ointments, pastes, mousses and cosmetics (e.g., solid, semi-solid, or liquid make-up, including foundations, eye-makeup, pigmented or non-pigmented lip treatments, e.g., lipsticks, and the like). These product forms may comprise several types of carriers including, but not limited to, solutions, aerosols, emulsions, gels, solids, and liposomes.

One exemplary carrier contains a dermatologically acceptable, hydrophilic diluent. As used herein, “diluent” includes materials in which the particulate material can be dispersed, dissolved, or otherwise incorporated. Nonlimiting examples of hydrophilic diluents are water, organic hydrophilic diluents such as lower monovalent alcohols (e.g., C1-C4) and low molecular weight glycols and polyols, including propylene glycol, polyethylene glycol (e.g., Molecular Weight 200-600 g/mole), polypropylene glycol (e.g., Molecular Weight 425-2025 g/mole), glycerol, butylene glycol, 1,2,4-butanetriol, sorbitol esters, 1,2,6-hexanetriol, ethanol, isopropanol, sorbitol esters, butanediol, ether propanol, ethoxylated ethers, propoxylated ethers and combinations thereof. Water is one preferred diluent.

An emulsion is another exemplary dermatologically acceptable carrier in accordance with the present invention. Emulsion carriers contain a hydrophilic phase comprising a hydrophilic component, e.g., water or other hydrophilic diluent, and a hydrophobic phase comprising a hydrophobic component, e.g., a lipid, oil or oily material. As well known to one skilled in the art, the hydrophilic phase will be dispersed in the hydrophobic phase, or vice versa, to form respectively hydrophilic or hydrophobic dispersed and continuous phases, depending on the composition ingredients. The emulsion may be or comprise (e.g., in a triple or other multi-phase emulsion) an oil-in-water emulsion or a water-in-oil emulsion such as a water-in-silicone emulsion. Oil-in-water emulsions typically comprise from about 1% to about 50% (preferably about 1% to about 30%) of the dispersed hydrophobic phase and from about 1% to about 98% (preferably from about 40% to about 90%) of the continuous hydrophilic phase; water-in-oil emulsions typically comprise from about 1% to about 98% (preferably from about 40% to about 90%) of the dispersed hydrophilic phase and from about 1% to about 50% (preferably about 1% to about 30%) of the continuous hydrophobic phase. The emulsion may also comprise a gel network, such as described in G. M. Eccleston, Application of Emulsion Stability Theories to Mobile and Semisolid O/W Emulsions, Cosmetics & Toiletries, Vol. 101, November 1996, pp. 73-92.

The compositions of the present invention, including but not limited to lotions and creams, may comprise an emollient. Such compositions preferably contain from about 2% to about 50% of the emollient. Emollients are typically water-immiscible, oily or waxy materials. A wide variety of suitable emollients are known and may be used herein. Sagarin, Cosmetics, Science and Technology, 2nd Edition, Vol. 1, pp. 32 43 (1972), contains numerous examples of materials suitable as an emollient.

Compositions of this invention useful for cleansing (“cleansers”) are formulated with a suitable carrier, e.g., as described above, and preferably contain one or more dermatologically acceptable surfactants in an amount which is safe and effective for cleansing. Preferred compositions contain from about 1% to about 90%, more preferably from about 5% to about 10%, of a dermatologically acceptable surfactant. The surfactant is suitably selected from anionic, cationic, nonionic, zwitterionic, amphoteric and ampholytic surfactants, as well as mixtures of these surfactants. Examples of a broad variety of surfactants useful herein are described in McCutcheon's Detergents and Emulsifiers, North American Edition (1986), published by Allured Publishing Corporation. The cleansing compositions can optionally contain, at their art-established levels, other materials which are conventionally used in cleansing compositions.

The compositions of the present invention are preferably formulated to have a pH of 10.5 or below. The pH values of these compositions preferably range from about 2 to about 10.5, more preferably from about 3 to about 8, even more preferably from about 5 to about 8.

One preferred dermatologically acceptable carrier is in the form of an oil-in-water emulsion.

The dermatologically acceptable carrier may contain other ingredients, such as thickening agents, structuring agents, silicone elastomers, and mixtures thereof (more fully discussed below) in order to modify the viscosity and/or feel of the composition.

Optional Ingredients

The compositions of the present invention may contain one or more additional skin care ingredients. In a preferred embodiment, where the composition is to be in contact with human keratinous tissue, the additional components should be suitable for application to keratinous tissue, that is, when incorporated into the composition they are suitable for use in contact with human keratinous tissue without undue toxicity, incompatibility, instability, allergic response, and the like within the scope of sound medical judgment.

The CTFA Cosmetic Ingredient Handbook, Second Edition (1992) describes a wide variety of nonlimiting cosmetic and pharmaceutical ingredients commonly used in the personal care industry, which are suitable for use in the compositions of the present invention.

Exemplary optional ingredients will be discussed below.

A. Silicone Elastomers

The compositions of the present invention may contain a silicone elastomer. When present, the composition preferably comprises from about 0.1% to about 30%, more preferably from about 1% to about 20%, and even more preferably, from about 2% to about 15%, by weight of the composition, of a silicone elastomer component.

The compositions of the present invention may include an emulsifying crosslinked organopolysiloxane elastomer, a non-emulsifying crosslinked organopolysiloxane elastomer, or a mixture thereof. The term “non-emulsifying,” as used herein, defines crosslinked organopolysiloxane elastomers from which polyoxyalkylene units are absent. The term “emulsifying,” as used herein, means crosslinked organopolysiloxane elastomers having at least one polyoxyalkylene (e.g., polyoxyethylene or polyoxypropylene) unit. No specific restriction exists as to the type of curable organopolysiloxane composition which can serve as starting material for the crosslinked organopolysiloxane elastomer.

Non-limiting examples of emulsifying elastomers include polyoxyalkylene modified elastomers formed from divinyl compounds, particularly siloxane polymers with at least two free vinyl groups, reacting with Si—H linkages on a polysiloxane backbone. Preferably, the elastomers are dimethyl polysiloxanes crosslinked by Si—H sites on a molecularly spherical MQ resin. Emulsifying crosslinked organopolysiloxane elastomer can notably be chosen from the crosslinked polymers described in U.S. Pat. Nos. 5,412,004 (issued May 2, 1995); 5,837,793 (issued Nov. 17, 1998); and 5,811,487 (issued Aug. 22, 1998). In addition, an emulsifying elastomer comprised of dimethicone copolyol crosspolymer (and) dimethicone is available from Shin Etsu under the tradename KSG-21.

Non-limiting examples of non-emulsifying elastomers are dimethicone/vinyl dimethicone crosspolymers. Such dimethicone/vinyl dimethicone crosspolymers are supplied by a variety of suppliers including Dow Corning (DC 9040 and DC 9041), General Electric (SFE 839), Shin Etsu (KSG-15, 16, 18 [dimethicone/phenyl vinyl dimethicone crosspolymer]), and Grant Industries (GRANSIL™ line of elastomers). Cross-linked organopolysiloxane elastomers useful in the present invention and processes for making them are further described in U.S. Pat. No. 4,970,252 to Sakuta, et al., issued Nov. 13, 1990; U.S. Pat. No. 5,760,116 to Kilgour, et al., issued Jun. 2, 1998; U.S. Pat. No. 5,654,362 to Schulz, Jr., et al. issued Aug. 5, 1997. Additional crosslinked organopolysiloxane elastomers useful in the present invention are disclosed in Japanese Patent Application JP 61-18708, assigned to Pola Kasei Kogyo KK.

Commercially available elastomers suitable for use herein include Dow Corning's 9040 silicone elastomer blend, Shin Etsu's KSG-21, and mixtures thereof.

B. Structuring Agent

Compositions of this invention may contain from about 0.1% to about 20%, more preferably from about 0.1% to about 10%, still more preferably from about 0.5% to about 9%, of one or more structuring agents.

Exemplary structuring agents suitable for use herein are those having an HLB of from about 1 to about 8, and having a melting point of at least about 45° C. Non-limiting examples of structuring agents useful in compositions of the present invention include stearic acid, palmitic acid, stearyl alcohol, cetyl alcohol, behenyl alcohol, stearic acid, palmitic acid, the polyethylene glycol ether of stearyl alcohol having an average of about 1 to about 5 ethylene oxide units, the polyethylene glycol ether of cetyl alcohol having an average of about 1 to about 5 ethylene oxide units, and mixtures thereof.

C. Thickening Agents

The compositions of the present invention may include one or more thickening agents. When present, the composition preferably includes from about 0.1% to about 5%, more preferably from about 0.1% to about 4%, and still more preferably from about 0.25% to about 3%, by weight of the composition of the thickening agent.

Nonlimiting examples of thickening agents useful herein include carboxylic acid polymers such as the carbomers (such as those commercially available under the tradename Carbopol® 900 series from B. F. Goodrich; e.g., Carbopol® 954). Other suitable carboxylic acid polymeric agents include copolymers of C₁₀₋₃₀ alkyl acrylates with one or more monomers of acrylic acid, methacrylic acid, or one of their short chain (i.e., C₁₋₄ alcohol) esters, wherein the crosslinking agent is an allyl ether of sucrose or pentaerytritol. These copolymers are known as acrylates/C10-30 alkyl acrylate crosspolymers and are commercially available as Carbopol® 1342, Carbopol® 1382, PEMULEN TR-1, and PEMULEN TR-2, from B. F. Goodrich.

Other nonlimiting examples of thickening agents include crosslinked polyacrylate polymers including both cationic and nonionic polymers. Still other nonlimiting examples of thickening agents include the polyacrylamide polymers, especially nonionic polyacrylamide polymers including substituted branched or unbranched polymers. More preferred among these polyacrylamide polymers is the nonionic polymer given the CTFA designation polyacrylamide and isoparaffin and laureth-7, available under the Tradename Sepigel 305 from Seppic Corporation (Fairfield, N.J.). Other polyacrylamide polymers useful herein include multi-block copolymers of acrylamides and substituted acrylamides with acrylic acids and substituted acrylic acids. Commercially available examples of these multi-block copolymers include HYPAN SR150H, SS500V, SS500W, SSSA100H, from Lipo Chemicals, Inc., (Patterson, N.J.).

Another nonlimiting class of thickening agents useful herein is polysaccharides. Nonlimiting examples of polysaccharide gelling agents include those selected from cellulose, and cellulose derivatives. Preferred among the alkyl hydroxyalkyl cellulose ethers is the material given the CTFA designation cetyl hydroxyethylcellulose, which is the ether of cetyl alcohol and hydroxyethylcellulose, sold under the tradename Natrosol® CS Plus from Aqualon Corporation (Wilmington, D.E.). Other useful polysaccharides include scleroglucans which are a linear chain of (1-3) linked glucose units with a (1-6) linked glucose every three units, a commercially available example of which is Clearogel™ CS11 from Michel Mercier Products Inc. (Mountainside, N.J.).

Another nonlimiting class of thickening agents useful herein is the gums. Nonlimiting examples of gums useful herein include hectorite, hydrated silica, xantham gum, and mixtures thereof.

D. Vitamins

An exemplary list of vitamins for inclusion herein includes vitamin B₃ compounds (such as niacinamide), Vitamin B₅ or derivatives (such as panthenol, pantothenoic acid), Vitamin E or derivatives (such as tocopherol, tocopherol acetate), or Vitamin D₃ or derivatives.

E. Proteins

Compositions of the present invention may comprise one or more proteins, such as, for example, hydrolyzed and non-hydrolyzed (i.e. “native”) animal and vegetable derived proteins. One particularly preferred protein is hydrolyzed wheat protein.

Plant derived non-hydrolyzed proteins useful herein, include, but are not limited to, soya proteins, wheat proteins, almond protein, potato protein, oat proteins, pea proteins, sun flower proteins, corn proteins, cottonseed proteins, peanut proteins, and wheat germ protein. Non-limiting examples of animal derived non-hydrolyzed proteins useful herein, include: milk proteins, such as β-lactoglobulin, casein, or whey; serum proteins, such as horse serum; placental proteins; albumen; amylase; collagen; crystalline; cytochrome C; elastin; fibronectin; gelatin; gliadin; keratin; lipase; and serum albumin.

F. Zeolites

Zeolites may be employed in compositions of the present invention, including, for example, natural zeolites such as analcite, chabazite, heulandite, natrolite, stilbite, and thomosonite; and synthetic zeolites such as those made by the gel process (sodium silicate and alumina) or a clay process (kaolin), which forms a matrix to which the zeolite is added.

G. Peptides

Peptides, including but not limited to, di-, tri-, tetra-, and pentapeptides and derivatives thereof, may be included in the compositions of the present invention in amounts that are safe and effective. Non-limiting examples of peptides and peptide derivatives useful herein include; Carnosine® ((beta-ala-his), gly-his-lys, arg-lys-arg, his-gly-gly, palmitoyl-gly-his-lys (which may be purchased as Biopeptide CL®, 100 ppm commercially available from Sederma, France), Peptide CK (arg-lys-arg), PEPTIDE CK+(ac-arg-lys-arg-NH2), and a copper derivative of his-gly-gly sold commercially as IAMIN, from Sigma (St. Louis, Miss.). Tetrapeptides and pentapeptides (such as palmitoyl-lys-thr-thr-lys-ser commercially available from Sederma France) are also suitable for use herein.

When included in the present compositions, peptides are preferably included in amounts of from about 1×10-6% to about 10%, more preferably from about 1×10-6% to about 0.1%, by weight of the composition.

H. Terpene Alcohols

Compositions of the present invention may, in some embodiments, contain a safe and effective amount of a terpene alcohol such as phytantriol, phytantriol derivatives, farnesol, farnesol derivatives, and mixtures thereof. When included in compositions of the present invention, the terpene alcohol is preferably is included in an amount from about 0.001% to about 50% by weight of the composition, more preferably from about 0.01% to about 20%, by weight of the composition.

I. Desquamation Actives

A safe and effective amount of a desquamation active may be added to the compositions of the present invention, preferably from about 0.1% to about 10%, more preferably from about 0.2% to about 5%, by weight of the composition. Non-limiting examples of desquamation systems useful herein include; a combination of sulfhydryl compounds and zwitterionic surfactants; and a combination of salicylic acid and zwitterionic surfactants.

J. Anti-Acne Actives

Compositions of the present invention may contain a safe and effective amount of one or more anti-acne actives. Examples of useful anti-acne actives include resorcinol, sulfur, salicylic acid, benzoyl peroxide, erythromycin, and zinc.

K. Anti-Wrinkle Actives/Anti-Atrophy Actives

The compositions of the present invention may contain a safe and effective amount of one or more anti-wrinkle actives or anti-atrophy actives. A representative, non-limiting examples of anti-wrinkle/anti-atrophy actives suitable for use in the compositions of the present invention include hydroxy acids (e.g., alpha-hydroxy acids such as lactic acid and glycolic acid or beta-hydroxy acids such as salicylic acid and salicylic acid derivatives such as the octanoyl derivative), phytic acid, lipoic acid; lysophosphatidic acid, and skin peel agents.

L. Flavonoids Compositions of the present invention may optionally contain a flavonoid compound. Flavonoids are broadly disclosed in U.S. Pat. Nos. 5,686,082 and 5,686,367. Non-limiting examples of flavonoids useful herein include unsubstituted flavone, 7,2′-dihydroxy flavone, 3′,4′-dihydroxy naphthoflavone, 4′-hydroxy flavone, 5,6-benzoflavone, and 7,8-benzoflavone, unsubstituted isoflavone, daidzein (7,4′-dihydroxy isoflavone), 5,7-dihydroxy-4′-methoxy isoflavone, soy isoflavones (a mixture extracted from soy), and mixtures thereof. When present, the flavonoid compounds are preferably present in concentrations of from about 0.01% to about 20%, more preferably from about 0.1% to about 10%, by weight of the composition.

M. Anti-Inflammatory Agents

A safe and effective amount of an anti-inflammatory agent may be added to the compositions of the present invention, preferably from about 0.1% to about 10%, more preferably from about 0.5% to about 5%, of the composition.

Nonlimiting examples of “natural” anti-inflammatory agents that are useful herein include candelilla wax, bisabolol (e.g., alpha bisabolol), aloe vera, plant sterols (e.g., phytosterol), and mixtures thereof. Additional anti-inflammatory agents useful herein include glycyrrhizinate compounds such as dipotassium glycyrrhizinate.

N. Anti-Cellulite Agents

Compositions of the present invention may also contain a safe and effective amount of an anti-cellulite agent. Exemplary anti-cellulite agents include xanthine compounds (e.g., caffeine, theophylline, theobromine, and aminophylline).

O. Topical Anesthetics

The compositions of the present invention may also contain a safe and effective amount of a topical anesthetic. Examples of topical anesthetic drugs include, but are not limited to, benzocaine, lidocaine, pharmaceutically acceptable salts thereof, and mixtures thereof.

P. Tanning Actives

The compositions of the present invention may contain a tanning active. When present, it is preferable that the compositions contain from about 0.1% to about 20%, more preferably from about 2% to about 7%, by weight of the composition, of the artificial tanning active. One exemplary tanning active useful herein is dihydroxyacetone.

Q. Skin Lightening Agents

A skin lightening agent may be employed in compositions of the present invention. When used, the compositions preferably contain from about 0.1% to about 10%, more preferably from about 0.2% to about 5%, by weight of the composition, of a skin lightening agent. Non-limiting examples of skin lightening agents useful herein include those known in the art, including niacinamide, kojic acid, arbutin, ascorbic acid and derivatives thereof (e.g., sodium ascorbyl phosphate), and extracts (e.g., mulberry extract, placental extract).

R. Skin Soothing and Skin Healing Actives

A safe and effective amount of a skin soothing or skin healing active may be added to the present composition, preferably, from about 0.1% to about 30%, more preferably from about 0.5% to about 20%, by weight of the composition. Skin soothing or skin healing actives suitable for use herein include, but are not limited to, panthenoic acid derivatives (including panthenol, dexpanthenol, ethyl panthenol), aloe vera, allantoin, bisabolol, and dipotassium glycyrrhizinate.

S. Conditioning Agent

Some embodiments of the present invention may contain a conditioning agent selected from humectants, moisturizers, or skin conditioners, for example. A variety of these materials can be employed and each can be present at a level of from about 0.01% to about 20%, more preferably from about 0.1% to about 10%, by weight of the composition. Conditioning agents useful herein include, but are not limited to, hyaluronic acid, glycerin, panthenol, allantoin, and mixtures thereof. Also useful are various C1-C30 monoesters and polyesters of sugars and related materials.

Compositions of the present invention are generally prepared by conventional methods such as are known in the art of making personal care compositions. Such methods typically involve mixing of the ingredients in one or more steps to a relatively uniform state, with or without heating, cooling, application of vacuum, and the like. The compositions are preferably prepared such as to optimize stability (physical stability, chemical stability, photostability) and/or delivery of the active materials. This optimization may include appropriate pH (e.g., less than 7), exclusion of materials that can complex with the active agent and thus negatively impact stability or delivery (e.g., exclusion of contaminating iron), use of approaches to prevent complex formation (e.g., appropriate dispersing agents or dual compartment packaging), and use of appropriate photostability approaches (e.g., incorporation of sunscreen/sunblock, use of opaque packaging).

At least some of the compositions of the present invention are useful for regulating keratinous tissue, particularly mammalian skin conditions. Such regulation of keratinous tissue conditions can include prophylactic and therapeutic regulation.

Examples of regulating skin conditions include, but are not limited to thickening keratinous tissue (i.e., building the epidermis and/or dermis layers of the skin and where applicable the keratinous layers of the nail and hair shaft), preventing and/or retarding atrophy of mammalian skin, preventing and/or retarding the appearance of spider vessels and/or red blotchiness on mammalian skin, treating (i.e. preventing and/or retarding the appearance of) dark circles under the eye of a mammal, preventing and/or retarding sallowness of mammalian skin, regulating (i.e. preventing and/or retarding) sagging of mammalian skin, softening and/or smoothing lips, hair and nails of a mammal, preventing and/or relieving itch of mammalian skin, regulating skin texture (e.g., wrinkles and fine lines), regulating the appearance of shiny skin, treating (i.e. preventing and/or retarding the appearance of) cellulite, increasing the rate of skin turnover, and improving skin color (e.g., redness, freckles).

Regulating keratinous tissue condition is preferably practiced by applying a composition in the form of a skin lotion, cream, gel, foam, ointment, paste, serum, stick, emulsion, spray, conditioner, tonic, cosmetic, lipstick, foundation, nail polish, after-shave, or the like to portions of the tissue. The compositions are preferably intended to be left on the keratin structure for some esthetic, prophylactic, therapeutic or other benefit (i.e., a “leave-on” composition). After applying the composition to the skin and/or hair, it is preferably left on the tissue for a period of at least about 15 minutes, more preferably at least about 30 minutes, even more preferably at least about 1 hour, still more preferably for at least several hours, e.g., up to about 12 hours. Any part of the external portion of the face, hair, and/or nails can be treated, e.g., face, lips, under-eye area, upper lip, eyelids, scalp, neck, torso, arms, underarms, hands, legs, feet, fingernails, toenails, scalp hair, eyelashes, eyebrows, etc. The composition can be applied with the fingers or with an implement or device (e.g., pad, cotton ball, applicator pen, spray applicator, and the like).

Another approach to ensure a continuous exposure of the tissue to at least a minimum level of the active is to apply the composition via a patch—e.g., to the face. Such an approach is particularly useful for problem skin areas needing more intensive treatment (e.g., facial crows feet area, frown lines, under eye area, upper lip and the like). The patch can be occlusive, semi-occlusive or non-occlusive and can be adhesive or non-adhesive. The composition can be contained within the patch or be applied to the skin prior to application of the patch. The patch can also include additional actives such as chemical initiators for exothermic reactions such as those described in U.S. Pat. Nos. 5,821,250; 5,981,547; and 5,972,957, to Wu, et al. The patch is preferably left on the skin for a period of at least about 5 minutes, more preferably at least about 15 minutes, more preferably still at least about 30 minutes, even more preferably at least about 1 hour, still more preferably at night as a form of night therapy.

In a preferred embodiment, the composition is chronically applied to the skin. By “chronic topical application” is meant continued topical application of the composition over an extended period during the subject's lifetime, preferably for a period of at least about one week, more preferably for a period of at least about one month, even more preferably for at least about three months, even more preferably for at least about six months, and more preferably still for at least about one year. Typically applications would be on the order of about once per day over such extended periods, however application rates can vary from about once per week up to about three times per day or more.

A wide range of quantities of the compositions of the present invention can be employed to provide an appearance and/or feel benefit. Quantities of the present compositions, which are typically applied per application, are, in mg composition/cm² tissue, from about 0.1 mg/cm² to about 20 mg/cm². An exemplary application amount is from about 0.5 mg/cm² to about 10 mg/cm².

Personal care compositions of the present invention have improved color stability. One manner of measuring the color stability of a composition is by determining the level of one or more color components manifested by the composition over a targeted shelf/use life time period (the time period can be simulated through accelerated aging) using, for example, a Macbeth color measurement device. One color level that is of particular interest is yellow since oxidation of vitamin C, for example, tends to result in a yellowing of compositions containing this active. Delta b is the level of yellowness that can be measured with a Macbeth color measurement device. Compositions of the present invention preferably have a delta b value of less than 2 at a six-month exposure time and exposure temperature of 40° C., and more preferably have a delta b value of less than 1 at the same criteria. Compositions having a delta b above 2 at a six-month exposure time are also contemplated herein.

### III. EXAMPLE

The following example further describes and demonstrates an embodiment within the scope of the present invention. The example is given solely for the purpose of illustration and is not to be construed as a limitation of the present invention, as many variations thereof are possible without departing from the spirit and scope of the invention.

Phase A

| Ingredient | Weight % |
|---|---|
| Yeast Ferment Filtrate (available from | 20.0 |
| Kashiwayama of Japan) | |
| Glycerin | 10.0 |
| Water (purified) | Quantity sufficient |
| Niacinamide | 3.5 |
| Ascorbyl glucoside | 2.0 |
| Propylene Glycol | 1.0 |
| Butylene Glycol | 1.0 |
| Panthenol, D form | 1.0 |
| Benzyl alcohol | 0.4 |
| Allantoin | 0.05 |
| Ethylparaben | 0.1 |
| Propylparaben | 0.1 |
| Disodium EDTA | 0.1 |
| Sodium hydroxide | 0.2750 |
| Sodium Metabisulfite | 0.015 |

Phase B

| Ingredient | Weight % |
|---|---|
| Cyclopentasiloxane | 22.5556 |
| Cyclopentasiloxane and Dimethicone/Vinyl | 15.5 |
| dimethicone crosspolymer | |
| Polyethylene | 7 |
| Dimethicone and PEG-10 Dimethicone | 2.5 |
| crosspolymer | |
| Ethylhexyl methoxycinnamate | 2.0 |
| DL alpha tocopheryl acetate | 0.5 |
| Titanium oxide and Cyclopentasiloxane | 0.4 |
| and Alumina and Dimethicone and | |
| PEG/PPG-18/18 Dimethicone | |
| Bis-PEG/PPG-14/14 Dimethicone and | 0.6 |
| Cyclopentasiloxane | |
| PEG-10 Dimethicone | 0.25 |

Phase C

| | |
|---|---|
| Beta-carotene and Zea Mays (corn) oil and | 0.0004 |
| Tocopherol | |

In a first vessel, mix the Phase A ingredients at room temperature, and then heat to 37° C. In a second vessel, mix the Phase B ingredients at room temperature until evenly mixed. Add the contents of the first vessel to the second vessel, and mix until an emulsification is achieved—approximately 5 minutes. Add Phase C to the second vessel, and mix until evenly mixed. Mix all of the contents in the second vessel with a homogenizer for approximately 10 minutes at 32° C. Cool the final composition to room temperature.

All cited documents are incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care composition, comprising:
a) an active that is unstable in oxidizing medium;
b) a color stabilizing system comprising a carotenoid, an anti-oxidant, and a UVB absorbing agent; and
c) a dermatologically acceptable carrier.

2. The personal care composition of claim 1, wherein the active comprises vitamin C and/or a derivative thereof.

3. The personal care composition of claim 1, further comprising vitamin B₃ and/or a derivative thereof.

4. The personal care composition of claim 1, wherein the color stabilizing system further comprises an inorganic pigment.

5. The personal care composition of claim 1, wherein the carotenoid comprises beta-carotene.

6. The personal care composition of claim 1, wherein the anti-oxidant comprises sodium metabisulfite.

7. The personal care composition of claim 1, wherein the UVB absorbing agent comprises octyl methoxycinnamate.

8. A personal care composition, comprising:
a) an active that is unstable in oxidizing medium;
b) a color stabilizing system comprising a carotenoid and an ultraviolet light absorbing agent; and
c) a dermatologically acceptable carrier.

9. The personal care composition of claim 8, wherein the active comprises vitamin C and/or a derivative thereof.

10. The personal care composition of claim 9, wherein the vitamin C and/or derivative thereof is ascorbyl glucoside.

11. The personal care composition of claim 8, wherein the carotenoid comprises beta-carotene.

12. The personal care composition of claim 8, wherein the ultraviolet light absorbing agent comprises octyl methoxycinnamate.

13. The personal care composition of claim 8, wherein the color stabilizing system further comprises an anti-oxidant.

14. The personal care composition of claim 13, wherein the anti-oxidant comprises sodium metabisulfite.

15. The personal care composition of claim 8, wherein the color stabilizing system further comprises an inorganic pigment.

16. The personal care composition of claim 15, wherein the inorganic pigment comprises titanium dioxide.

17. The personal care composition of claim 15, wherein the inorganic pigment has a particle size of less than about 1.0 micron.

18. The personal care composition of claim 15, wherein the inorganic pigment is coated with a material selected from the group consisting of a silicone, aluminum stearate, and combinations thereof.

19. The personal care composition of claim 8, further comprising vitamin B₃ and/or a derivative thereof.

20. The personal care composition of claim 19, wherein the vitamin B₃ and/or derivative thereof comprises niacinamide.

21. The personal care composition of claim 8, wherein the composition is a water-in-silicone emulsion system.

22. The personal care composition of claim 8, wherein the composition comprises water in an amount of less than about 30% by weight of the composition.

23. A method for stabilizing color of a personal care composition containing an active that is unstable in oxidizing medium, comprising the step of:
incorporating into the composition a color stabilizing system comprising a carotenoid and an ultraviolet light absorbing agent.

24. The method of claim 23, wherein the color stabilizing system further comprises an inorganic pigment.

25. The method of claim 23, wherein the color stabilizing system further comprises an anti-oxidant.

26. A method for stabilizing color of a personal care composition comprising vitamin C and/or a derivative thereof, comprising the step of:
incorporating into the composition a color stabilizing system comprising a carotenoid; a UVB absorbing agent; and an anti-oxidant.

27. The method of claim 26, wherein the vitamin C and/or a derivative thereof comprises ascorbyl glucoside.

28. The method of claim 26, wherein the catotenoid comprises beta-carotene.

29. The method of claim 26, wherein the UVB absorbing agent comprises octyl methoxycinnamate.

30. The method of claim 26, wherein the color stabilizing system further comprises an inorganic pigment.

31. A method for stabilizing color of a personal care composition that turns yellow when exposed to oxidizing medium, comprising the step of:
incorporating into the personal care composition a natural yellow, orange, red, and/or brown pigment; and
incorporating into the personal care composition two or more materials that inhibit oxidation selected from the group comprising anti-oxidants, inorganic pigments, and ultraviolet light absorbing agents.
